(19) United States
(12) Patent Application Publication
(10) Pub. No.: US 2006/0122229 A1
(43) Pub. Date: Jun. 8, 2006

(54) 4,5-diarylthiazole derivatives as cb-1 ligands

(75) Inventors: Anna Ingrid Kristina Berggren, Molndal (SE); Stig Jonas Bostrom, Monlndal (SE); Stig Thomas Elebring, Molndal (SE); Linda Fallefors, Molndal (SE); Johan Michael Wilstermann, Molndal (SE); Peter Greasley, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/538,318

(22) PCT Filed: Dec. 18, 2003
(86) PCT No.: PCT/GB03/05542

§ 371 Date: Jun. 13, 2005

(30) Foreign Application Priority Data
Dec. 24, 2002 (GB) 02300879

(51) Int. Cl. A61K 31/454; A61K 31/427; A61K 31/426; C07D 417/02

(52) U.S. Cl. 514/326; 514/369; 546/208; 548/181; 548/200

(57) ABSTRACT

The present invention relates to compounds of formula (I): 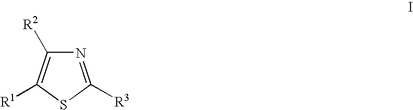 in which R¹ and R² independently represent phenyl, thienyl or pyridyl and R³ represents a group —X—Y—NR⁴R⁵ in which X is CO or SO₂; Y is absent or represents NH and the other substituente are as defined in the description and their use in the treatment of obesity, psychiatric and neurological disorders and to pharmaceutical compositions containing them.

### FIELD OF INVENTION

The present invention relates to certain 4,5-diarylthiazole-2-carboxamide compounds, to processes for preparing such compounds, to their use in the treatment of obesity, psychiatric and neurological disorders, and to pharmaceutical compositions containing them.

### BACKGROUND OF THE INVENTION

It is known that certain CB, modulators (known as antagonists or inverse agonists) are useful in the treatment of obesity, psychiatric and neurological disorders (WO01/70700 and EP 656354). However, there is a need for CB, modulators with improved physicochemical properties and/or DMPK (distribution, metabolism and pharmacokinetic) properties and/or pharmacodynamic properties.

Certain N-acyl-4,5-diarylthiazoles-2-alkylamines and N-acyl-4,5-diarylthiazoles-2-carboxamides are reported to have antithrombotic activity in EP388909 and EP 377457. Other such thiazoles are disclosed in British Journal of Pharmacology (2002), 135(3), 782-788; European Journal of Pharmacology (2000), 391(1/2), 49-54 ;Bioorganic & Medicinal Chemistry (1999), 7(8), 1559-1565; WO9420475; WO9420476; Journal of Medicinal Chemistry (1994), 37(8), 1189-99; Journal of Pharmacology (1993), 243(2), 179-84; European Journal of Pharmacology (1993 Oct. 19), 243(2), 179-84; and the Journal of Medicinal Chemistry (1994 Apr. 15), 37(8), 1189-99. The compounds disclosed in these documents are disclaimed from the compound claims of the present invention.

### DESCRIPTION OF THE INVENTION

The invention relates to compounds of the general formula (1) 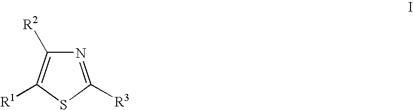
and pharmaceutically acceptable salts, prodrugs and solvates thereof, in which
R¹ and R² independently represent phenyl, thienyl or pyridyl each of which is optionally substituted by one, two or three groups represented by Z;
Z represents a C₁₋₆alkyl group, a C₁₋₆alkoxy group, hydroxy, halo, trifluoromethyl, trifluoromethylthio, trifluoromethoxy, trifluoromethylsulphonyl, nitro, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl, acetyl or two adjacent carbons may be substituted with the group —O—CH₂—CH₂—O—; and phenyl optionally substituted by one or more of the following: C₁₋₆alkyl group, trifluoromethyl, a C₁₋₆alkoxy group, trifluoromethoxy, or halo or two adjacent carbons may be substituted with the group —O—CH₂—CH₂—O—; and
R³ represents a group —X—Y—NR⁴R⁵ in which
R⁴ and R⁵ independently represent:
a C₁₋₆alkyl group optionally substituted by a C₁₋₆alkoxy group or trifluoromethoxy;
an (amino)C₁₋₄alkyl- group in which the amino is optionally substituted by one or more C₁₋₃alkyl groups;
a non-aromatic C₃₋₅carbocyclic group which is optionally substituted by a C₁₋₃alkoxyC₁₋₃alkyl group;
a (C₃₋₁₂cycloalkyl)C₁₋₃allyl- group;
a group —CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
naphthyl;
anthracenyl;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups or benzyl; 1-adamantylmethyl;
a group —(CH₂)ₜ Het in which t is 0, 1, 2, 3 or 4, and the alkylene chain is optionally substituted by one or more C₁₋₃alkyl groups and Het represents an aromatic heterocycle optionally substituted by one, two or three groups selected from a C₁₋₆alkyl group; a C₁₋₆alkoxy group, trifluoromethoxy or halo or Het represents a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
or R⁴ represents H and R⁵ is as defined above;
or R⁴ and R⁵ together with the nitrogen atom to which they are attached represent a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
X is CO or SO₂;
Y is absent or represents NH optionally substituted by a C₁₋₃alkyl group; with the proviso that R¹ and R² do not both represent 4-methoxyphenyl and the proviso that
when R¹ represents phenyl and R² represents phenyl or 4-fluorophenyl, X is CO and Y is absent then the group NR⁴R⁵ does not represent methyl-[2-[1-(phenylmethyl)-4-piperidinyl]ethyl]amino, methylpiperazino, 2-[1-methyl-4-piperidinyl]ethylamino; or [2-[1-(phenylmethyl)-4-piperidinyl]ethyl]amino.

Further values of R¹, R² and R³ in compounds of formula I now follow. It will be understood that such values may be used where appropriate with any of the definitions, claims or embodiments defined hereinbefore or hereinafter.

In one group of compounds of formula I, R¹ represents phenyl optionally substituted by one or two halos, particularly chloro or bromo, or by a C₁₋₃alkoxy group.

In a second group of compounds of formula I, R¹ represents a 2,3-dihydrobenzo[1,4]dioxinyl group optionally substituted by one or more halo.

In a third group of compounds of formula I, R¹ represents phenyl, 4-chlorophenyl, 4-bromophenyl, 4-methoxyphenyl, 2,4 dichlorophenyl or 7-bromo-2,3-dihydrobenzo[1,4]dioxin-6-yl.

In a fourth group of compounds of formula I, R² represents phenyl optionally substituted by one or two halos, particularly chloro or bromo, or by a C₁₋₃alkoxy group.

In a fifth group of compounds of formula I, R² represents a 2,3-dihydrobenzo[1,4]dioxinyl group optionally substituted by one or more halo.

In a sixth group of compounds of formula I, R² represents phenyl, 4-chlorophenyl, 4-bromophenyl, 4-methoxyphenyl, 2,4 dichlorophenyl or 7-bromo-2,3-dihydrobenzo[1,4]dioxin-6-yl.

In a seventh group of compounds of formula I, X is CO, Y is absent and R³ represents a C₃₋₇cycloalkylamino group.

In an eighth group of compounds of formula I, X is CO, Y is absent and R³ represents pyridylamino.

In an ninth group of compounds of formula I, X is CO, Y is absent and R³ represents a C₁₋₆alylamino group wherein the alkyl chain is substituted by one or more of the following: a C₁₋₃alkoxy group, or morpholino.

In a tenth group of compounds of formula I, X is CO, Y is absent and R³ represents cyclohexylamino, piperidin-1-ylamino, (2-methoxymethylcyclopentyl)amino, pyridin-4-ylamino, (2-ethoxyethyl)amino; or (2-(morpholin-4-yl)ethyl)amino.

One group of compounds of formula I is represented by formula (II) 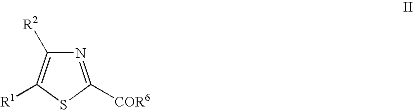
and pharmaceutically acceptable salts, prodrugs and solvates thereof, in which R¹ represents phenyl optionally substituted by one or more of the following: C₁₋₆alkyl group, trifluoromethyl, a C₁₋₆alkoxy group, trifluoromethoxy, or halo or two adjacent carbons may be substituted with the group —O—CH₂—CH₂—O—,
R² represents phenyl optionally substituted by one or more of the following: C₁₋₃alkyl group, trifluoromethyl, a C₁₋₆alkoxy group, trifluoromethoxy, or halo or two adjacent carbons may be substituted with the group —O—CH₂—CH₂—O—, and
R⁶ represents 1-piperidinylamino, a C₃₋₇cycloalkylamino group which is optionally substituted is by a C₁₋₃alkoxyC₁₋₃alkyl group, pyridylamino wherein the pyridyl ring is optionally substituted by one or more of the following: a C₁₋₆alkyl group; a C₁₋₆aloxy group or trifluoromethoxy; or R⁶ represents a C₁₋₆alkylamino group wherein the alkyl chain is optionally substituted by one or more of the following: a C₁₋₆alkoxy group, trifluoromethoxy or morpholino;
with the proviso that when R¹ represents 4-methoxyphenyl and R² represents 4 methoxyphenyl then R⁶ does not represent 2-(morpholino)ethyl.

Further values of R¹, R² and R⁶ in compounds of formula II now follow. It will be understood that such values may be used where appropriate with any of the definitions, claims or embodiments defined hereinbefore or hereinafter.

In one group of compounds of formula II, R¹ represents phenyl optionally substituted by one or two halos, particularly chloro or bromo, or by a C₁₋₃alkoxy group.

In a second group of compounds of formula II, R¹ represents a 2,3-dihydrobenzo[1,4]dioxinyl group optionally substituted by one or more halo.

In a third group of compounds of formula II, R¹ represents phenyl, 4-chlorophenyl, 4-bromophenyl, 4-methoxyphenyl, 2,4 dichlorophenyl or 7-bromo-2,3-dihydrobenzo[1,4]dioxin-6-yl.

In a fourth group of compounds of formula II, R² represents phenyl optionally substituted by one or two halos, particularly chloro or bromo, or by a C₁₋₃alkoxy group.

In a fifth group of compounds of formula II, R² represents a 2,3-dihydrobenzo[1,4]dioxinyl group optionally substituted by one or more halo.

In a sixth group of compounds of formula II, R² represents phenyl, 4-chlorophenyl, 4-bromophenyl, 4-methoxyphenyl, 2,4 dichlorophenyl or 7-bromo-2,3-dihydrobenzo[1,4]dioxin-6-yl.

In a seventh group of compounds of formula II, R⁶ represents a C₃₋₇cycloalkylamino group.

In an eighth group of compounds of formula II, R⁶ represents pyridylamino.

In an ninth group of compounds of formula II, R⁶ represents a C₁₋₆alkylamino group wherein the alkyl chain is substituted by one or more of the following: a C₁₋₃alkoxy group, or is morpholino.

In a tenth group of compounds of formula I, R¹ represents cyclohexylamino, piperidin-1-ylamino, (2-methoxymethylcyclopentyl)amino, pyridin-4-ylamino, (2-ethoxyethyl)amino; or (2-(morpholin-4-yl)ethyl)amino.

“Pharmaceutically acceptable salt”, where such salts are possible, includes both pharmaceutically acceptable acid addition salts. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid;

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention.

The following definitions shall apply throughout the specification and the appended claims. Unless otherwise stated or indicated, the term “alkyl” denotes either a straight or branched alkyl group. Examples of said alkyl include methyl, ethyl, n-propyl, isopropyl, n-butyl, isobutyl, sec-butyl and t-butyl . Preferred allkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

Specific compounds of the invention are:
4-(4-chlorophenyl)-5-(2,4-dichlorophenyl)thiazole-2-carboxylic acid cyclohexylamide;
5-(4-chlorophenyl)-4-(2,4-dichlorophenyl)thiazole-2-carboxylic acid cyclohexylamide;
4-(4-chlorophenyl)-5-(2,4-dichlorophenyl)thiazole-2-carboxylic acid piperidin-1-ylamide;
5-(4chlorophenyl)-4-(2,4-dichlorophenyl)thiazole-2-carboxylic acid piperidin-1-ylamide;
4-(4-bromophenyl)-5-phenylthiazole-2-carboxylic acid cyclohexylamide;
4-(4-bromophenyl)-5-phenylthiazole-2-carboxylic acid piperidin-1-ylamide;
4,5-bis-(4-chlorophenyl)thiazole-2-carboxylic acid cyclohexylamide;
4,5-bis-(4-chlorophenyl)thiazole-2-carboxylic acid piperidin-1-ylamide;
4-(4-methoxyphenyl)-5-phenylthiazole-2-carboxylic acid cyclohexylamide;
4,5-bis-(4-methoxyphenyl)thiazole-2-carboxylic acid cyclohexylamide;
4,5-bis-(4-methoxyphenyl)thiazole-2-carboxylic acid piperidin-1-ylamide;
5-(7-bromo-2,3-dihydrobenzo[1,4]dioxin-6-yl)-4-phenylthiazole-2-carboxylic acid piperidin-1-ylamide;
4-(7-bromo-2,3-dihydrobenzo[1,4]dioxin-6-yl)-5-phenylthiazole-2-carboxylic acid piperidin-1-ylamide;
4,5-bis-(4-chlorophenyl)thiazole-2-carboxylic acid (2-methoxymethylcyclopentyl)amide;
4,5-bis-(4-chlorophenyl)thiazole-2-carboxylic acid pyridin-4-ylamide;
4,5-bis-(4-chlorophenyl)thiazole-2-carboxylic acid (2-ethoxyethyl)amide; and
4,5-bis-(4-chlorophenyl)thiazole-2-carboxylic acid (2-morpholinyl-ethyl)amide and where applicable, optical isomers, tautomers, stereoisomers and racemates thereof as well as pharmaceutically acceptable salts and solvates thereof.

It should be understood that the present invention includes each of the above compounds and any combination of two or more these compounds that is 2, 3, 4, 5, 6, 7, 8, 9, 10, 11, 12, 13, 14, 15, 16 or 17 of these compounds.

Methods of Preparation

The compounds of the invention may be prepared as outlined below according to any of the following methods. However, the invention is not limited to these methods, the compounds may also be prepared as described for structurally related compounds in the prior art. Compounds of formula I in which X is CO may be prepared by reacting a compound of formula III 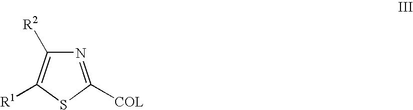
in which R¹, and R² are as previously defined and L represents hydroxy, alkoxy or halo (particularly chloro or bromo) with an amine of formula IV
R⁴R⁵NYH₂ IV
in which R⁴ and R⁵ are as previously defined in an inert solvent, for example dichloromethane, in the presence of a coupling agent, for example a carbodiimide, eg 1-(3-dimethylamino-propyl)-3-ethylcarbodiimide, and optionally in the presence of a catalyst, for example a basic catalyst, eg 4-dimethylaminopyridine, at a temperature in the range of −25° C. to 150° C.

Compounds of formula m may be prepared as described in the Examples and by other methods known to those skilled in the art. Certain compounds of formula II are novel and are claimed as a further aspect of the present invention as useful intermediates.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

The expression “inert solvent” refers to a solvent which does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free acid, or a pharmaceutically acceptable organic or inorganic base addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

The compounds of the invention may also be combined with other therapeutic agents which are useful in the treatment of disorders associated with obesity.

A compound of the invention may also be combined with other anti-obesity agents such as Orlistat or a monoamine reuptake inhibitor, for example Sibutramine. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

According to a further aspect of the invention there is also provided a pharmaceutical formulation including any of the compounds of the invention, or pharmaceutically acceptable derivatives thereof, in admixture with pharmaceutically acceptable adjuvants, diluelits and/or carriers.

Pharmacological Properties

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders(e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight which normally accompanies the cessation of smoking.

In another aspect the present invention provides a compound of formula I as previously defined for use as a medicament.

In a further aspect the present invention provides the use of a compound of formula I (including the compounds of the proviso) in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I including the compounds of the proviso to a patient in need thereof.

The compounds of the present invention are particulary suitable for the treatment of obesity, e.g. by reduction of appetite and body weight, maintenance of weight reduction and prevention of rebound.

Combination Therapy

The compounds of the invention may be combined with another therapeutic agent that is useful in the treatment of disorders associated with the development and progress of obesity such as hypertension, hyperlipidaemias, dyslipidaemias, diabetes and atherosclerosis. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound;
an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;
a Melanin concentrating hormone (MCH) antagonist;
a PDK inhibitor; or
modulators of nuclear receptors for example TXR, FXR, RXR, and RORalpha;
an SSRI;
a serotonin antagonist;
   or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

### GENERAL EXPERIMENTAL PROCEDURES

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300, Varian Unity plus 400 or a Varian INOVA 500, operating at ¹H frequencies of 300, 400 and 500 MHz respectively. Chemical shifts are given in ppm with CDCl₃ as internal standard if nothing else stated. Purification was performed by semipreparative HPLC if nothing else stated. Two different semipreparative HPLC systems were used:
(a) The Shimadzu system was equipped with a Waters, xTerra 19×100 mm C₁₈, 5 μm column and a QP 8000 single quadrupole mass spectrometer. The fraction collector was mass triggered. The mobile phase used was acetonitrile and buffer (0.1 M NH₄OAc:acetonitrile 95:5).
(b) The Waters Prep LC 2000 system was equipped with a HICHROM, 21.1×250 mm C₈, 7 μm column. The system was equipped with a UV detector (Waters 2487 Dual λ Absorbance Detector). The mobile phase used was acetonitrile and buffer (0.1 M NH₄OAc:acetonitrile 95:5).

Microwave heating was performed using single node heating in a Smith Creator or Smith Synthesizer from Personal Chemistry, Uppsala, Sweden.

### LIST OF ABBREVIATIONS

DCM dichloromethane
t triplet
s singlet
d doublet
q quartet
m multiplet
br broad
dd doublet of doublet
p pentet
   Synthesis of Intermediates

Preparation A

(a) 2-Bromo-2-(4-chlorophenyl)-1-(2,4-dichlorophenyl)ethanone

Bromine (1 M in acetic acid, 4.66 ml, 4.66 nmol) was added dropwise to 2-(4-chlorophenyl)-1-(2,4-dichlorophenyl)ethanone (1.27 g, 4.23 mmol) dissolved in acetic acid (15 ml) with stirring at room temperature. After stirring at room temperature for 2.5 hours an additional portion of bromine (0.2 eq, 1 M in acetic acid) was added and the mixture was stirred for an additional 3.5 hours. Water (50 ml) was added and the solution was extracted with DCM, dried (MgSO₄), filtered and evaporated under reduced pressure to give the crude product (1.59 g, 99%). ¹H-NMR (500 MHz) δ 7.49-7.45 (m, 3H), 7.42-7.31 (m, 4H), 6.19 (s, 1H). MS m/z 375, 377, 379, 381 (M−H)⁻.

(b) 2-Bromo-2-(7-bromo-2,3-dihydro-benzo[1,4]dioxin-6-yl)-1-phenylethanone

2-(7-Bromo-2,3-dihydrobenzo[1,4]dioxin-6-yl)-1-phenylethanone (500 mg, 1.50 mmol) was dissolved in acetic acid (7 ml) and treated with bromine (263 mg, 1.65 mmol) as described in

Preparation A step (a). After 5 hours, the reaction mixture was worked up as described in Preparation A step (a) to give the crude product (576 mg, 93%). MS m/z 409, 411, 413 (M−)⁻.

Preparation B

Starting materials for Preparation B were either commercially available or described in Preparation A.

(a) 4-(4-Chlorophenyl)-5-(2,4-dichlorophenyl)thiazole-2-carboxylic acid ethyl ester or 5-(4-Chlorophenyl)(2,4-dichloronhenyl)thiazole-2-carboxylic acid ethyl ester

Ethyl thiooxamate (75 mg, 0.56 mmol) was added to a solution of 2-bromo-2-(4-chlorophenyl)-1-(2,4-dichloro-phenyl)ethanone (212 mg, 0.56 mmol) from preparation A step (a) in ethanol (10 mL). The mixture was subjected to microwave heating 120° C. for 80 minutes. The solvent was evaporated under reduced pressure and cold acetonitrile was added to the residue. The precipitate was filtered off, the solution concentrated and the residue chromatographed (SiO₂, heptane:ethyl acetate 5:1) to give one of the title compounds (43.5 mg, 19%). ¹H-NMR (400 MHz) δ7.42 (d, 1H), 7.36 (d, 1H), 7.30-7.26 (m, 3H), 7.16 (m, 2H), 4,50 (q, 2H), 1.45 (t, 3M). MS m/z 412, 414, 416 (M+H)⁺.

(b) 5-(4-Chlorophenyl)-4-(2,4-dichlorophenyl)thiazole-2-carboxylic acid ethyl ester or 4-(4-Chloroohenyl)-5-(2,4-dichlorophenyl)thiazole-2-carboxhlic acid ethyl ester

Ethyl thiooxamate (76 mg, 0.58 mmol) was added to a solution of 2-bromo-2-(4-chlorophenyl)-1-(2,4dichlorophenyl)ethanone (220 mg, 0.58 mmol) from preparation A step (a) in ethanol (10 mL). The mixture was subjected to microwave heating at 150° C. for 20 minutes. The solvent was evaporated under reduced pressure, cold acetonitrile was added to the residue. The product precipitated and was filtered off as white solid (53.8 mg, 22%). ¹H-NMR (C₃D₇NO, 400 MHz), 8.38 (d, 1H), 7.88 (d, 11), 7.75-7.67 (m, 3H), 7.64-7.58 (m, 2H), 4.28 (q, 2H), 1.21 (t, 3H). MS 7m/z 412, 414, 416 (M+H)⁺.

(c) 4-(4-Bromophenyl)-5-phenyl-thiazole-2-carboxylic acid ethyl ester

Ethyl thiooxamate (167 mg, 1.26 mmol) was added to a solution of 2-bromo-1-(4-bromophenyl)-2-phenyl-ethanone (578 mg, 1.16 mmol) in ethanol (25 ml). The mixture was subjected to microwave heating 150° C. for 20 minutes. The solvent was evaporated under reduced pressure, chloroform was added and the precipitate formed was filtered off. The concentrated residue was chromatographed (SiO₂, heptane:ethyl acetate 9:1) to give the title compound (272 mg, 60%). ¹H-NMR (400 MHz) δ 7.48-7.38 (m, 9H), 4,55 (q, 2H), 1.51 (t, 3H). MS m/z 389 (M+H)⁺.

(d) 4,5-Bis-(4-chlorophenyl)thiazole-2-carboxylic acid ethyl ester

Ethyl thiooxamate (203 mg, 1.52 mmol) was added to a solution of 2-bromo-1,2-bis-(4-chlorophenyl)ethanone (525 mg, 1.07 mmol) in ethanol (25 ml). The mixture was subjected to microwave heating at 150° C. for 10 minutes. An additional 0.13 eq. of ethyl thiooxamate was added, and the mixture was heated for another 5 minutes at 150° C. using microwave heating. The solvent was evaporated under reduced pressure, chloroform was added and the precipitate formed was filtered off. The concentrated residue was chromatographed (SiO₂, heptane:ethyl acetate 9:1) to give the title compound (233 mg, 58%). ¹H-NMR (500 MHz) δ 7.48 (m, 2H), 7.39 (m, 2H), 7.34-7.30 (m, 4H), 4,54 (q, 2H), 1.49 (t, 3H). MS m/z 378, 380, 382 (M+H)⁺.

(e) 4,5-Bis-(4-methoxyphenyl)thiazole-2-carboxylic acid ethyl ester

Ethyl thiooxamate (195 mg, 1.46 mmol) was added to a solution of 2-bromo-1,2-bis-(4-methoxyphenyl)ethanone (490 mg, 1.46 mmol) in ethanol (25 ml). The mixture was subjected to microwave heating 150° C. for 30 minutes. The solvent was evaporated under reduced pressure. Heptane:ethyl acetate (5:1) was added to the residue and undissolved impurities were filtered off before the residue was concentrated and chromatographed (SiO₂, heptane:ethyl acetate 5:1) to give the impure title compound (317 mg, 52% purity, 31%). MS m/z 370 (M+H)⁺. The impure material was taken to the next step without further purification.

(f) 5-(7-Bromo-2,3-dihydrobenzo[1,4[dioxin-6-yl)-4-phenylthlazole-2-carboxhlic acid ethyl ester and 4-(7-Bromo-2,3-dihydrobenzo[1,4]dioxin-6-yl)-5-phenylthiazole-2-carboxylic acid ethyl ester

2-Bromo-2-(7-bromo-2,3-dihydrobenzo[1,4]dioxin-6-yl)-1-phenylethanone (400 mg, 0.97 mmol) from Preparation A step (b) was treated as described in Preparation B step (a) but heated to 150° C. for 1 hour using microwave heating. Purification by semipreparatory HPLC system (a) gave the two title compounds (30 mg, 6.8%) and (22 mg, 5.0%). ¹H-NMR (300 MHz) δ 7.30 (s, 5H), 7.08 (s, 1H), 6.93 (s, 1H), 4,50 (q, 2H), 4.26 (q, 4H), 1.45 (t, 3H) and δ 7.76 (s, 1H), 7.57-7.53 (m, 2H), 7.46-7.41 (m, 3H), 7.18 (s, 1H), 4.33-4.26 (m, 6H), 1.24 (t, 3H).

Preparation C

(a) 5-(4-Chloro-phenyl)-4-(2,4-dichlorophenyl)-thiazole-2-carboxylic acid or 4-(4-Chloro-phenyl)-5-(2,4-dichlorophenyl)-thiazole-2-carboxylic acid

Sodium hydroxide (109 mg, 2.73 mmol) was added to a solution of 5-(4-chloro-phenyl)-4-(2,4-dichlorophenyl)thiazole-2-carboxylic acid ethyl ester or 4-(4-chloro-phenyl)-5-(2,4-dichlorophenyl)thiazole-2-carboxylic acid ethyl ester (75.0 mg, 0.18 nmol) from preparation B step (b) in ethanol (3 mL). The mixture was refluxed for 2 hours, then allowed to reach room temperature and the solvent was evaporated under reduced pressure. Hydrochloric acid (aq, 2 M, 25 ml) was added and the mixture was stirred overnight. The solution was extracted with ethyl acetate, the combined organic phases were washed with brine, dried (MgSO₄), filtered and concentrated under reduced pressure to give the crude title compound (68 mg, 97 %). MS m/z 384, 386, 388 (M+H)⁺. The crude product was used in steps described below without further purification.

(b) 4,5-Bis-(4-chlorophenyl)thiazole-2-carboxylic acid 4,5-Bis-(4-chlorophenyl)thiazole-2-carboxylic acid ethyl ester (486 mg, 1.28 mmol) from Preparation B step (d) was treated as described in Preparation C step (a) but refluxed for 30 minutes. The reaction mixture was worked up as described in Preparation C step (a) but was not stirred overnight to give the title compound (434 mg, 97 to) MS m/z 350, 352, 354 (M+H)⁺. The crude product was used without further purification. Examples of the invention

### EXAMPLE 1

### 4-(4-Chlorophenyl)-5-(2,4-dichlorophenyl)thiazole-2-carboxylic acid cyclohexylamide or 5-(4-Chlorophenyl)-4-(2,4-dichlorophenyl)thiazole-2-carboxylic acid cyclohexylamide

4-(4-Chlorophenyl)-5-(2,4-dichlorophenyl)thiazole-2-carboxylic acid ethyl ester or 5-(4-Chlorophenyl)-4-(2,4-dichlorophenyl)thiazole-2-carboxylic acid ethyl ester (24 mg, 0.058 mmol) from Preparation B step (a) was dissolved in cyclohexylamine (3 mL, 26.2 mmol) and the mixture was subjected to microwave heating at 150° C. for 15 minutes. The solution was evaporated under reduced pressure and the residue was chromatographed (SiO₂, heptane:ethyl acetate 9:1) to give the title compound (24 mg, 82%). ¹H-NMR (400 MHz) δ 7.46 (d, 1H), 7.31-7.24 (m, 3H), 7.15-7.11 (m, 2H), 7.07 (d, 1H), 3.95 (m, 1H), 2.02 (m, 2H), 1.77 (m, 2H), 1.62 (m, 1H), 1.48-1.16 (m, 5H). MS m/z 463, 465, 467, 469(M+H)⁺.

### EXAMPLE 2

### 4-(4-Chlorophenyl)-5-(2,4-dichlorophenyl)thiazole-2-carboxylic acid Riperidin-1-ylamide or 5-(4-Chlorophenyl)-4-(2,4-dichlorophenyl)thiazole-2-carboxylic acid piperidin-1-ylamide

4-(4-Chlorophenyl)-5-(2,4-dichlorophenyl)thiazole-2-carboxylic acid ethyl ester or 5-(4-Chlorophenyl)-4-(2,4-dichlorophenyl)thiazole-2-carboxylic acid ethyl ester (42 mg, 0.10 mmol) from Preparation B step (a) was dissolved in N-aminopiperidine (3 mL, 27.8 mmol) and the mixture was subjected to microwave heating at 150° C. for 30 minutes. The solution was evaporated under reduced pressure and the residue was chromatographed (SiO₂, toluene:ethyl acetate 1:0→5:1) to give the title compound (24 mg, 51%). ¹H-NMR (500 MHz) δ 7.94 (s, 1H), 7.47 (m, 1H), 7.32-7.25 (m, 4H), 7.14 (m, 2H), 2.89 (m, 4H), 1.77 (m, 4 Hz), 1.45 (m, 2H). MS m/z 466, 468, 470 (M+H)⁺.

### EXAMPLE 3

### 5-(4-Chlorophenyl)-4-(2,4-dichlorophenyl)thiazole-2-carboxylic acid Riperidin-1-ylamide or 4-(4-Chlorophenyl)-5-(2,4-dichlorophenylthiazole-2-carboxylic acid piperidin-1-ylamide

5-(4-Chlorophenyl)-4-(2,4dichlorophenyl)thiazole-2-carboxylic acid or 4-(4-chlorophenyl)-5-(2,4dichlorophenyl)thiazole-2-carboxylic acid (51 mg, 0.13 mmol) from Preparation C step (a) and 4-dimethylaminopyridine (2 mg, 0.013 mmol) were dissolved in DCM (9 ml) and DMF (0.5 ml). The solution was cooled to 0° C. A slurry of 1-ethyl-3-(3-dimethylaminopropyl)carbodiimide hydrochloride (32 mg, 0.16 mmol) in DCM (0.5 ml) was added dropwise. After 15 minutes N-aminopiperidine (16 μl, 0.15 mmol) in DCM (0.5 ml) was added dropwise. The mixture was allowed to attain room temperature, and was stirred overnight. The mixture was diluted with DCM, washed with NaHCO₃ (aq), dried (MgSO₄) and evaporated under reduced pressure. The residue was chromatographed (SiO₂, toluene:ethyl acetate 9:1) to give the title compound (20 mg, 31%). ¹H-NMR (500 MHz) δ 8.21 (d, 1H), 7.64 (d, 2H), 7.55 (d, 1H), 7.41 (dd, 1H), 7.38 (d, 2H), 2.96 (br, 411), 1.77 (br, 4H), 1.46 (br, 2H). MS m/z 466, 468, 470 (M+H)⁺.

### EXAMPLE 4

### 4-(4-Bromophenyl)-5-phenylthiazole-2-carboxylic acid cyclohexylamide 4-(4-Bromophenyl)-5-phenylthiazole-2-carboxylic acid ethyl ester (52 mg, 0.14 mmol) from

Preparation B step (c) was dissolved in cyclohexylamine (2 ml, 17.5 mmol) and the mixture 15 was subjected to microwave heating at 150° C. for 10 minutes. The solvent was evaporated under reduced pressure and the residue was chromatographed (SiO₂, toluene) to give the title compound (40 mg, 68%). ¹H-NMR (400 M) δ 7.44 (m, 2H), 7.39-7.31 (m, 7H), 2.04 (m, 2H), 1.78 (m, 2H), 1.66 (m, 1H), 1.49-1.16 (m, 5H). MS mi/z 441, 443 (M+H)⁺.

### EXAMPLE 5

### 4-(4-Bromophenyl)-5-phenylthiazole-2-carboxlic acid piperidin-1-ylamide

4-(4-Bromophenyl)-5-phenylthiazole-2-carboxylic acid ethyl ester (27 mg, 0.070 mmol) from

Preparation B step (c) was dissolved in N-aminopiperidine (1.5 ml, 13.9 mmol) and the mixture was subjected to microwave heating at 150° C. for 25 minutes. The solution was evaporated under reduced pressure and chromatographed (SiO₂, toluene:ethyl acetate 5:1) to give the title compound (14 mg, 45%). ¹H-NMR (400 MHz) δ 7.99 (s, 1H), 7.44 (m, 2H), 7.39-7.30 (m, 71), 2.91 (m, 4H), 1.78 (m, 4H), 1.47 (m, 2H). MS m/z 442, 444 (M+H)⁺.

### EXAMPLE 6

### 4,5-Bis-(4-chlorophenyl thiazole-2-carboxylic acid cyclohexylamide

4,5-Bis-(4-chlorophenyl)thiazole-2-carboxylic acid ethyl ester (50 mg, 0.13 mmol) from Preparation B step (d) was dissolved in cyclohexylamine (3 ml, 26.2 mmol) and the mixture was subjected to microwave heating at 180° C. for 30 minutes. The solution was evaporated under reduced pressure and the residue was chromatographed (SiO₂, toluene:ethyl acetate 19:1) to give the title compound (53 mg, 93%). ¹H-NMR (400 MHz) δ 7.42 (m, 2H), 7.35-7.22 (m, 6H), 3.95 (m, 1H), 2.04 (m, 2H), 1.78 (m, 2H), 1.66 (m, 1H), 1.49-1.16 (m, 5H). MS m/z 431, 433, 435 (M+H)⁺.

### EXAMPLE 7

### 4,5-Bis-(4-chlorophenyl)thiazole-2-carboxylic acid piperidin-1-ylamide

4,5-Bis-(4-chlorophenyl)thiazole-2-carboxylic acid ethyl ester (55 mg, 0.14 mmol) from Preparation B step (d) was dissolved in N-aminopiperidine (2 ml, 18.5 mmol) and the mixture was subjected to microwave heating at 150° C. for 30 minutes. The solution was evaporated under reduced pressure and the residue was chromatographed (SiO₂, toluene:ethyl acetate 19:1→5:1) to give the title compound (26 mg, 41%). ¹H-NMR (400 MHz) δ 7.98 (bs, 1H), 7.41 (m, 2H), 7.36-7.22 (m, 6H), 2.91 (m, 4H), 1.78 (m, 4H), 1.47 (m, 2H). MS m/z 432, 434, 436 (M+H)⁺.

### EXAMPLE 8

### 4-(4-Methoxyphenyl)-5-phenylthiazole-2-carboxylic acid cyclohexylamide

4-(4-Methoxyphenyl)-5-phenylthiazole-2-carboxylic acid ethyl ester (51 mg, 0.15 mmol) was dissolved in cyclohexylamine (4 ml, 35.0 mmol) and the mixture was subjected to microwave heating at 180° C. for 20 minutes. The solution was evaporated under reduced pressure and the residue was chromatographed twice (SiO₂, toluene: ethyl acetate 19:1 then SiO₂, toluene:ethyl acetate 5:1) to give the title compound (37 mg, 62%). ¹H-NMR (400 MHz) α 7.43 (m, 2H), 7.34 (m, 4H), 7.18 (m, 1H), 6.84 (m, 2H), 3.96 (m, 1H), 3.82 (s, 3H), 2.03 (m, 2H), 1.78 (m, 2H), 1.66 (m, 1H), 1.49-1.16 (m, 5H). MS m/z 393 (M+H)⁺.

### EXAMPLE 9

### 4,5-Bis-(4-methoxyphenyl)thiazole-2-carboxylic acid cyclohexylamide

The crude 4,5-bis-(4-methoxyphenyl)thiazole-2-carboxylic acid ethyl ester (54 mg, 0.03 mmol) from Preparation B step (e) was dissolved in cyclohexylamine (3 ml, 26.2 mmol) and the mixture was subjected to microwave heating at 180° C. for 2 hours. The solution was evaporated under reduced pressure and the residue was purified by semipreparative HPLC system (b) to give the title compound (26 mg, 81%). ¹H-NMR (400 MHz) 87.44 (m, 2H), 7.27 (m, 2H), 6.88-6.82 (m, 4H), 3.96 (m, 1H), 3.81 (s, 6H), 2.03 (m, 2H), 1.77 (m, 2H), 1.65 (m, 1H), 1.49-1.16 (m, 5H). MS m/z 423 (M+H)⁺.

### EXAMPLE 10

### 4,5-Bis-(4-methoxyphenl)thiazole-2-carboxylic acid Riiperidin-1-ylamide

The crude 4,5-Bis-(4-methoxyphenyl)thiazole-2-carboxylic acid ethyl ester (58 mg, 0.08 mmol) from Preparation B step (e) was dissolved in N-aminopiperidine (3 ml, 27.8 mmol) and the mixture was subjected to microwave heating at 150° C. for 3 hours. The solution was evaporated under reduced pressure and the residue was chromatographed (SiO₂, heptane:ethyl acetate 3:1). The product was not completely pure and another purification by semi-preparative HPLC system (b) gave the title compound (12 mg, 36%). ¹H-NMR (400 MHz) δ 7.43 (m, 2H), 7.26 (m, 2H), 6.88-6.82 (m, 4H), 3.83 (s, 6H), 3.68 (br, 4H), 1.82 (m, 4H), 1.49 (m, 2H). MS m/z 424 (4+H)⁺.

### EXAMPLE 11

### 5-(7-Bromo-2,3-dihydrobenzo[1,4]dioxin-6-yl)-4-phenylthiazole-2-carboxylic acid piperidin-1-ylamide or 4-(7-Bromo-2,3-dihydrobenzor[1,4]dioxin-6-yl)-5-phenyl-thiazole-2-carboxylic acid piperidin-1-ylamide

5-(7-Bromo-2,3-dihydrobenzo[1,4]dioxin-6-yl)-4-phenythiazole-2-carboxylic acid ethyl ester or 4-(7-Bromo-2,3-dihydrobenzo[1,4]dioxin-6-yl)-5-phenyl-thiazole-2-carboxylic acid ethyl ester (29 mg, 0.065 mmol) from Preparation B step (f) was treated and worked-up as described in Example 2. Flash chromatography (SiO₂, hexane:ethyl acetate 2:1) gave the title compound (13 mg, 40%). ¹H-NMR (300 MHz) δ 7.97 (s, 1H), 7.33-7.23 (m, 5H), 7.13 (s, 1H), 6.88 (s, 1H), 4.27 (m, 4H), 2.87 (m, 4H), 1.76 (p, 4H) 1.49-1.38 (m, 2H). MS m/z 500, 502 (M+H)⁺.

### EXMAPLE 12

### 4,5-Bis-(4-chlorophenyl)thiazole-2-carboxylic acid (2-methoxymethylcyclopentyl)amide

The title compound was isolated when 4,5-Bis-(4-chlorophenyl)thiazole-2-carboxylic acid ethyl ester (100 mg, 264 mmol) from Preparation B step (d) was treated with (R)-(+)-2-(methoxymethyl)-1-pyrrolidinamine (2 ml) as described in Example 1 at 180° C. for 15 minutes. Purification by flash chromatography twice (SiO₂, 1% methanol in DCM then SiO₂, 2.5% methanol in DCM) gave the title compound (3 mg, 2.5%). ¹H NMR (300 MHz) δ 7.47-7.28 (m, 8H), 4,5 (m, 1H), 4.22 (t, 2H), 3.71 (m, 2H), 3.37 (s, 3H), 2.10-1.91 (m, 4H). MS m/z 447, 449, 451 (M+H)⁺.

### EXAMPLE 13

### 4,5-Bis-(4-chlorophenyl)thiazole-2-carboxylic acid pyridin-4-ylamide

4,5-Bis-(4-chlorophenyl)thiazole-2-carboxylic acid (400 mg, 1.14 mmol) from Preparation C step (b) was dissolved in toluene and thionyl chloride (816 mg, 6.86 mmol) was added. The reaction mixture was boiled under reflux for 3 hours. Solvent and excess of thionyl chloride were removed by evaporation under reduced pressure and the residue was dissolved in DCM (16 ml). The solution was divided into eight portions and one of these portions was stirred with 4-aminopyridine (15 mg, 0.16 mmol) and triethylamine (29 mg, 0.29 mmol) at room temperature overnight. The solvent was evaporated under reduced pressure and the residue was purified by flash chromatography (SiO₂, toluene then ethyl acetate) to give the title compound (5 mg, 8%, calculated on ⅛ of the starting material). ¹H NMR (500 MHz) δ 9.60 (s, 1H), 8.55 (d, 2H), 7.93 (d, 2H), 7.64 (m, 2H), 7.52 (d, 2H), 7.47 (d, 2H). MS m/z 426, 428, 430 (M+H)⁺.

### EXAMPLE 14

### 4,5-Bis-(4-chlorophenyl)thiazole-2-carboxylic acid (2-ethoxyethyl)amide

4,5-Bis-(4-chlorophenyl)thiazole-2-carboxylic acid ethyl ester (110 mg, 0.291 mmol) from Preparation B step (d) was dissolved in 2-ethoxyethylamine (2 ml) and treated as described in Example 1. Chromatography (SiO₂, 1% methanol in DCM) gave the title compound (77 mg, 63%). ¹H NMR (300 MHz) δ 7.43 (d, 2H), 7.36-7.25 (m, 6H), 3.71-3.60 (m, 4H), 3.55 (q, 2H), 1.24 (t, 3H). MS m/z 421, 423, 425 (M+H)⁺.

### EXAMPLE 15

### 4,5-Bis-(4-chlorophenyl)thiazole-2-carboxylic acid (2-morpholinyl-ethyl)amide

4,5-Bis-(4-chlorophenyl)thiazole-2-carboxylic acid ethyl ester (127 mg, 0.235 mmol) from Preparation B step (d) was dissolved in 2-(4-morpholino)ethylamine (2 ml) and treated as described in Example 1. Filtration through a Silica plug with methanol as eluent and then flash chromatography (SiO₂, 5% methanol in DCM) gave the title compound (54 mg, 50%). ¹H NMR (300 MHz) δ 7.43 (d, 2H), 7.38-7.23 (m, 6H), 3.74 (b, 4H), 3.63-3.55 (m, 2H), 2.62 (t, 2H), 2.53 (br, 4H). MS m/z 462, 464, 466 (M+H)⁺.

Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 nM NaCl, 5MM MgCl₂, 1mM EDTA, 50mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5nMM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x) UD)) and the IC50 value determined as the concentration required to give half maximal inhibition of GTPγS binding under the conditions used.

The compounds of the present invention are active at the CB1 receptor (IC50 <1 micromolar). Most preferred compounds have IC50 <200 nanomolar.

1. A compound of formula (I) 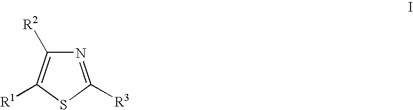
wherein
R¹ and R² are independently selected from phenyl, thienyl, and pyridyl, each of which is independently optionally substituted with one, two or three Z groups;
Z is selected from a C₁₋₆alkyl group, a C₁₋₆alkoxy group, hydroxy, halo, trifluoromethyl, trifluoromethylthio, trifluoromethoxy, trifluoromethylsulphonyl, nitro, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl, acetyl, —O—CH₂—CH₂—O— attached at two adjacent carbons, and phenyl, optionally substituted with one or more of the following: a C₁₋₆alkyl group, trifluoromethyl, a C₁₋₆alkoxy group, trifluoromethoxy, halo, or —O—CH₂—CH₂—O— attached at two adjacent carbons;
R³ is —X—Y—NR⁴R⁵, in which
R⁴ and R⁵ are independently selected from:
a C₁₋₆alkyl group, optionally substituted with a C₁₋₆alkoxy group or trifluoromethoxy; an (amino)C₁₋₄alkyl-group, wherein the amino is optionally substituted by one or more C₁₋₃alkyl groups;
a non-aromatic C₃₋₁₅carbocyclic group, optionally substituted with a C₁₋₃alkoxy C₁₋₃alkyl group;
a (C₃₋₁₂cycloalkyl)C₁₋₃alkyl- group;
a —(CH₂)ᵣ(phenyl)ₛ group, wherein r is 0, 1, 2, 3 or 4, and wherein s is 1 when r is 0, otherwise s is 1 or 2, and wherein the phenyl groups are optionally independently substituted with one, two or three Z groups;
naphthyl;
anthracenyl;
a saturated 5- to 8-membered heterocyclic group containing one nitrogen and optionally containing one of the following: oxygen, sulphur or an additional nitrogen, wherein the heterocyclic group is optionally substituted by one or more C₁₋₃alkyl groups or benzyl;
1-adamantylmethyl; and
a CH₂)ₜHet group, wherein t is 0, 1, 2, 3 or 4, and the alkylene chain is optionally substituted by one or more C₁₋₃alkyl groups, and wherein Het is an aromatic heterocycle optionally substituted by one, two or three groups selected from a C₁₋₆alkyl group; a C₁₋₆alkoxy group, trifluoromethoxy or halo or Het is a saturated 5- to 8-membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
and wherein R⁴ may be H;
and wherein R⁴ and R⁵ taken together with the nitrogen atom to which they are attached form a saturated 5- to 8-membered heterocyclic group containing one nitrogen and optionally one of the following: oxygen, sulphur or an additional nitrogen; wherein the heterocyclic group is optionally substituted with one or more C₁₋₃alkyl groups, hydroxy or benzyl;
X is CO or SO₂; and
Y is absent or represents NH optionally substituted by a C₁₋₃alkyl group;
or a pharmaceutically acceptable salt prodrug or solvate thereof;
with the proviso that R¹ and R² are not both represent 4-methoxyphenyl and the proviso that when R¹ is phenyl and R² represents phenyl or 4-fluorophenyl, X is CO and Y is absent then the group NR⁴R⁵ is not represent methyl-[2-[1-(phenylmethyl)-4-piperidinyl]ethyl]amino, methylpiperazino, 2-[1-methyl-4-piperidinyl]ethylamino, or [2-[1-(phenylmethyl)-4-piperidinyl]ethyl]amino.

2. A compound of formula I as represented by formula (II) 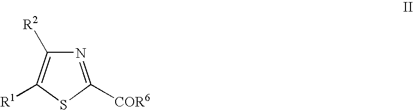
wherein
R¹ represents is phenyl optionally substituted by one or more of the following: a C₁₋₆alkyl group, trifluoromethyl, a C₁₋₆alkoxy group, trifluoromethoxy, halo, or —O—CH₂—CH₂—O— attached at two adjacent carbons;
R² is phenyl, optionally substituted by one or more of the following: a C₁₋₆alkyl group, trifluoromethyl, a C₁₋₄alkoxy group, trifluoromethoxy, of halo, or —O—CH₂—CH₂—O— attached at two adjacent carbons; and
R⁶ is selected from 1-piperidinylamino, a C₃₋₇cycloalkylamino group, optionally substituted by a C₁₋₃alkoxyC₁₋₃alkyl, pyridylamino, wherein the pyridyl ring is optionally substituted by one or more of the following: a C₁₋₆alkyl group; a C₁₋₆alkoxy group or trifluoromethoxy; a C₁₋₆alkylamino group, wherein the alkyl chain is optionally substituted by one or more of the following: a C₁₋₆alkoxy group, trifluoromethoxy or morpholino;
or a pharmaceutically acceptable salt, prodrug or solvate thereof:
with the proviso that when R¹ is 4-methoxyphenyl and R² is 4-methoxyphenyl, then R⁶ is not 2-(morpholino)ethyl.

3. A compound selected from:
4-(4-chlorophenyl)-5-(2,4-dichlorophenyl)thiazole-2-carboxylic acid cyclohexylamide;
5-(4-chlorophenyl)-4-(2,4-dichlorophenyl)thiazole-2-carboxylic acid cyclohexylamide;
4-(4-chlorophenyl)-5-(2,4-dichlorophenyl)thiazole-2-carboxylic acid piperidin-1-ylamide;
5-(4-chlorophenyl)-4-(2,4-dichlorophenyl)thiazole-2-carboxylic acid piperidin-1-ylamide;
4-(4-bromophenyl)-5-phenylthiazole-2-carboxylic acid cyclohexylamide;
4-(4-bromophenyl)-5-phenylthiazole-2-carboxylic acid piperidin-1-ylamide;
4,5-bis-(4-chlorophenyl)thiazole-2-carboxylic acid cyclohexylamide;
4,5-bis-(4-chlorophenyl)thiazole-2-carboxylic acid piperidin-1-ylamide;
4-(4-methoxyphenyl)-5-phenylthiazole-2-carboxylic acid cyclohexylamide;
4,5-bis-(4-methoxyphenyl)thiazole-2-carboxylic acid cyclohexylamide;
4,5-bis-(4-methoxyphenyl)thiazole-2-carboxylic acid piperidin-1-ylamide;
5-(7-bromo-2,3-dihydrobenzo[1,4]dioxin-6-yl)-4-phenylthiazole-2-carboxylic acid piperidin-1-ylamide;
4-(7-bromo-2,3-dihydrobenzo[1,4]dioxin-6-yl)-5-phenylthiazole-2-carboxylic acid piperidin-1-ylamide;
4,5-bis-(4-chlorophenyl)thiazole-2-carboxylic acid (2-methoxymethylcyclopentyl)-amide;
4,5-bis-(4-chlorophenyl)thiazole-2-carboxylic acid pyridin-4-ylamide;
4,5-bis-(4-chlorophenyl)thiazole-2-carboxylic acid (2-ethoxyethyl)amide; and
4,5-bis-(4-chlorophenyl)thiazole-2-carboxylic acid (2-morpholin-4-yl-ethyl)amide and where applicable, optical isomers, tautomers, stereoisomers and racemates thereof as well as pharmaceutically acceptable salts and solvates thereof.

4. (canceled)

5. A pharmaceutical formulation comprising a compound of any one of claims 1 to 3 and a pharmaceutically acceptable adjuvant, diluent or carrier.

6. (canceled)

7. A method of treating a condition selected from obesity, psychiatric disorders, psychotic disorders schizophrenia and bipolar disorders, anxiety, anxio-depressive disorders, depression, cognitive disorders, memory disorders, obsessive-compulsive disorders, anorexia, bulimia, attention disorders, ADHD, epilepsy, and related conditions, neurological disorders, dementia, neurological disorders, Multiple Sclerosis, Parkinson's Disease, Huntington's Chorea, Alzheimer's Disease, immune, cardiovascular, reproductive and endocrine disorders, septic shock, diseases related to the respiratory and gastrointestinal systems, diarrhea, extended abuse, addiction and/or relapse indications, drug (nicotine, ethanol, cocaine, opiates dependence, and drug (nicotine, ethanol, cocaine, opiates withdrawal symptoms in a mammal, comprising administering a pharmacologically effective amount of a compound as of formula (I) 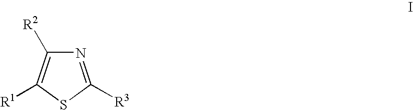
wherein
R¹ and R² are independently selected from phenyl, thienyl, and pyridyl, each of which is independently optionally substituted with one, two or three Z groups:
Z is selected from a C₁₋₄alkyl group, a C₁₋₆alkoxy group, hydroxy, halo, trifluoromethyl, trifluoromethylthio, trifluoromethoxy, trifluoromethylsulphonyl, nitro, amino, mono or di C₁₋₃alkylamino, mono or di C₁alkylamido, C₁₋₃alkylsulphonyl, C₁₋₃alkoxycarbonyl, carboxy, cyano, carbamoyl, mono or di C₁₋₃alkyl carbamoyl, sulphamoyl, acetyl, —O—CH₂—CH₂—O— attached at two adjacent carbons, and phenyl, optionally substituted with one or more of the following: a C₁₋₆alkyl group, trifluoromethyl, a C₁₋₆alkoxy group, trifluoromethoxy, halo, or —O—CH₂—CH₂—O— attached at two adjacent carbons:
R³ is —X—Y—NR⁴R⁵:
R⁴ and R⁵ are independently selected from:
a C₁₋₆alkyl group, optionally substituted with a C₁₋₆alkoxy group or trifluoromethoxy;
an (amino)C₁₋₄alkyl- group, wherein the amino is optionally substituted by one or more C₁₋₃alkyl groups;
a non-aromatic C₃₋₁₅carbocyclic group, optionally substituted with a C₁₋₃alkoxyC₁₋₃alkyl group;
a (C₃₋₁₂cycloalkyl)C₁₋₃alkyl- group;
a —(CH₂)ᵣ(phenyl), group, wherein r is 0, 1, 2, 3 or 4, and wherein s is 1 when r is 0, otherwise s is 1 or 2; and wherein the phenyl groups are optionally independently substituted with one, two or three Z groups:
naphthyl:
anthracenyl:
a saturated 5- to 8-membered heterocyclic group containing one nitrogen and optionally containing one of the following: oxygen, sulphur or an additional nitrogen wherein the heterocyclic group, is optionally substituted by one or more C₁₋₃alkyl groups or benzyl;
1-adamantylmethyl; and
a —(CH₂)ₜHet group, wherein t is 0, 1, 2, 3 or 4, and the alkylene chain is optionally substituted by one or more C₁₋₃alkyl groups, and wherein Het is an aromatic heterocycle optionally substituted by one, two or three groups selected from a C₁₋₆alkyl group; a C₁₋₆alkoxy group, trifluoromethoxy or halo or Het is a saturated 5- to 8-membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
and wherein R⁴ may be H;
and wherein R⁴ and R⁵ taken together with the nitrogen atom to which they are attached form a saturated 5- to 8-membered heterocyclic group containing one nitrogen and optionally one of the following: oxygen, sulphur or an additional nitrogen: wherein the heterocyclic group is optionally substituted with one or more C₁₋₃alkyl groups, hydroxy or benzyl;
X is CO or SO₂; and
Y is absent or represents NH optionally substituted by a C₁₋₃alkyl group;
or a pharmaceutically acceptable salt, prodrug or solvate thereof.

8. A process for the preparation of a compounds of claim 1 in which X is CO
comprising reacting a compound of formula III 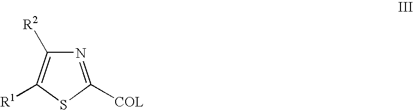
in which R¹, and R² are as previously defined and wherein L is hydroxy, alkoxy or halo with an amine of formula IV
R⁴ R⁵NYH₂ IV
in which Y, R⁴ and R⁵ are as previously defined in an inert solvent in the presence of a coupling agent and optionally in the presence of a catalyst at a temperature in the range of −25° C. to 150° C.

9. A compound of formula II selected from:
4-(4-Chlorophenyl)-5-(2,4-dichlorophenyl)thiazole-2-carboxylic acid ethyl ester,
5-(4-Chlorophenyl)-4-(2,4-dichlorophenyl)thiazole-2-carboxylic acid ethyl esters
4-(4-Bromophenyl)-5-phenyl-thiazole-2-carboxylic acid ethyl ester,
4,5-Bis-(4-chlorophenyl)thiazole-2-carboxylic acid ethyl esters
5-(7-Bromo-2,3-dihydrobenzo[1,4]dioxin-6-yl)-4-phenylthiazole-2-carboxylic acid ethyl ester,
4-(7-Bromo-2,3-dihydrobenzo[1,4]dioxin-6-yl)-5-phenylthiazole-2-carboxylic acid ethyl ester,
5-(4-Chloro-phenyl)-4-(2,4-dichlorophenyl)-thiazole-2-carboxylic acid,
4-(4-Chloro-phenyl)-5-(2,4-dichlorophenyl)-thiazole-2-carboxylic acid, and
4,5-Bis-(4-chlorophenyl)thiazole-2-carboxylic acid.

10. The composition according to claim 5, additionally comprising an agent useful for the treatment of hypertension, hyperlipidaemias, dyslipidaemias, diabetes or atherosclerosis.

11. A method of treating obesity, schizophrenia and bipolar disorders, anxiety, anxio-depressive disorders, depression, cognitive disorders, memory disorders, obsessive-compulsive disorders, anorexia, bulimia, ADHD, epilepsy, and related conditions, dementia, Multiple Sclerosis, Parkinson's Disease, Huntington's Chorea and Alzheimer's Disease, immune, cardiovascular, reproductive and endocrine disorders, septic shock, diarrhea, drug (nicotine, ethanol, cocaine, opiates) dependence, and drug (nicotine, ethanol, cocaine, opiates) withdrawal symptoms in a mammal comprising administering a pharmacologically effective amount of a compound of either of claims 2 or 3.
